(19) United States
(12) Patent Application Publication
(10) Pub. No.: US 2015/0297680 A1
(43) Pub. Date: Oct. 22, 2015

(54) COMPOSITIONS AND METHODS FOR INCREASING PROTEIN HALF-LIFE

(71) Applicant: University of Virginia Patent Foundation, Charlottesville, VA (US)

(72) Inventors: Inchan Kwon, Charlottesville, VA (US); Sung In Lim, Charlottesville, VA (US)

(73) Assignee: UNIVERSITY OF VIRGINIA PATENT FOUNDATION, Charlottesville, VA (US)

(21) Appl. No.: 14/677,798

(22) Filed: Apr. 2, 2015

Related U.S. Application Data
(60) Provisional application No. 61/974,007, filed on Apr. 2, 2014.

(51) Int. Cl. A61K 38/22; A61K 38/18; A61K 38/21; A61K 38/19; A61K 38/20

(52) U.S. Cl. CPC A61K 38/22; A61K 38/191; A61K 38/20; A61K 38/21; A61K 38/18

(57) ABSTRACT

In order to extend the serum half-life of a protein, we exploited the site-specific fatty acid-conjugation to a permissive site of a protein, using copper-catalyzed alkyne-azide cycloaddition, by linking a fatty acid derivative to p-ethynylphenylalanine incorporated into a protein using an engineered pair of yeast tRNA/aminoacyl tRNA synthetase. As a proof-of-concept, we show that single palmitic acid conjugated to superfolder green fluorescent protein (sfGFP) in a site-specific manner enhanced a protein's albumin-binding in vitro about 20 times and the serum half-life in vivo 5 times when compared to those of the unmodified sfGFP. Furthermore, the fatty acid conjugation did not cause a significant reduction in the fluorescence of sfGFP. Therefore, these results clearly indicate that the site-specific fatty acid-conjugation is a very promising strategy to prolong protein serum half-life in vivo without compromising its folded structure and activity.

### CROSS REFERENCE TO RELATED APPLICATIONS

This application is entitled to priority pursuant to 35 U.S.C. §119(e) to U.S. provisional patent application No. 61/974,007, filed on Apr. 2, 2014. The entire disclosure of the afore-mentioned patent application is incorporated herein by reference.

### BACKGROUND

Recombinant proteins with therapeutic activity have become critical for treating numerous diseases, and cover a wide range of therapeutics including monoclonal antibodies, hormones, growth factors, cytokines, and enzymes. However, utility of therapeutic proteins is often hampered by their short serum half-life requiring frequent re-administration resulting in patient discomfort and noncompliance. Therefore, extending the serum half-life of therapeutic proteins will significantly enhance the utility of existing therapeutic proteins and will also enable development of new therapeutic proteins. In the quest to extend the serum half-life, binding/conjugation of serum albumin or Fc portion of immunoglobulin G to therapeutic proteins is a very promising emerging strategy.

Human serum albumin (HSA) has an inherently long serum half-life (19 days) due to neonatal Fc receptor (FcRn)-mediated recycling as well as reduced renal filtration. HSA-binding/conjugation is a very attractive strategy for extending the serum half-life of a therapeutic protein when compared to conventional poly(ethylene)glycol (PEG) conjugation which mainly relies on renal filtration evasion. Furthermore, although it has long been considered that PEG is non-immunogenic, antibodies raised against PEG were observed in patients administered PEGylated uricase. Therefore, the binding of therapeutic proteins to HSAs in patients' blood is actively investigated to mitigate most immune response issues. Despite the many benefits of HSA as a binding/conjugation partner, developing a general strategy to bind/conjugate therapeutic proteins to HSA remains a big challenge.

In order to facilitate HSA binding of therapeutic proteins in patients' blood, genetic fusion of an albumin-binding domain to N-term or C-term of therapeutic proteins is performed. But this methodology has a potential risk of immunogenicity. Furthermore, the end-to-end fusion does not provide steric control or favorable topology that retains both therapeutic efficacy and conformational stability. Alternatively, synthetic or natural albumin-binding moieties have been chemically attached to a peptide, preferably to cysteine or lysine residues. In particular, the conjugation of a natural HSA ligand, a fatty acid, has been successfully used to extend the serum half-life in vivo of two therapeutic peptides, insulin, and glucagon-like peptide-1 agonist (GLP-1) via acylation at lysine residues. Seven binding sites in HSA have been identified to accommodate saturated fatty acids with 10-18 carbons. Compared to direct fusion/chemical conjugation of HSA to therapeutic proteins, this approach is advantageous for deep penetration into tissues, higher activity to mass ratio, and greatly reduced immunogenicity. However, fatty acid-conjugation to multiple lysine residues of therapeutic proteins likely leads to heterogeneous mixtures of the conjugated proteins, compromising pharmaceutical activity and downstream processing. For instance, fatty acid-conjugation to lysine residues of interferon-alpha led to an 80% reduction in its antiviral potency. Therefore, fatty acid-conjugation has been limited to peptides with a small number of lysine residues.

There is a long felt need in the art for compositions and methods useful for increasing the half-life of proteins in vivo, particularly for proteins being administered as therapeutic agents. The present invention satisfies these needs.

### SUMMARY OF THE INVENTION

The present application discloses compositions and methods for increasing protein and peptide half-life in vivo. In an effort to extend half-life of proteins and peptides in vivo, particularly in the circulation, binding/conjugation of serum albumin or Fc portion of immunoglobulin G to a protein or peptide of interest is a very promising emerging strategy. To overcome the heterogeneity of the conjugated proteins and the compromised pharmaceutical activity resulting from previously used methods, the present invention instead encompasses the use of a fatty acid attached to a permissive site of a protein necessitating site-specific fatty acid-conjugation techniques. In one aspect, the fatty acid is palmitic acid.

The present application discloses compositions and methods useful for site-specific conjugation of a fatty acid to a protein of interest, such as a therapeutic protein or peptide, to allow more predictable and uniform control of the fatty acid-protein once conjugated to HSA and to increase and provide more consistency and predictability of half-life of the fatty-acid-protein-HSA conjugate when administered to a subject.

One of ordinary skill in the art will appreciate that the compositions and methods of the invention can be modified for use in non-human animals as well by using an appropriate species of serum albumin or other proteins found in serum and an appropriate protein or peptide of interest for therapeutic purposes. By appropriate is meant a protein or peptide which is useful for the specific injury, disease, or disorder of the subject.

The therapeutic protein or peptide can be any protein or peptide which is useful for treating an injury, disease, or disorder. In one aspect, a pharmaceutical composition comprising an effective amount of modified protein or peptide is administered to a subject in need thereof. In one aspect, the protein or peptide has been modified to contain a nonstandard amino acid or non-canonical (NAA). In one aspect, the protein or peptide is conjugated to a fatty acid. In one aspect, the conjugation is site specific with the incorporated nonstandard amino acid. In one aspect, the fatty acid-protein/peptide is conjugated to serum albumin or another protein such as an antibody or fragment thereof. In one aspect, the pharmaceutical composition further comprises a pharmaceutically-acceptable carrier. In one aspect, the pharmaceutical further comprises at least one additional therapeutic agent.

Although therapeutic proteins are dominant over therapeutic peptides in clinical applications, to our knowledge, fatty acid-conjugation to a protein in a site-specific manner has not been reported. The present applications discloses a novel strategy to achieve site-specific conjugation of the natural albumin ligand, a fatty acid, to a protein with the combined use of copper-catalyzed alkyne-azide cycloaddition (CuAAC) and site-specific incorporation of noncanonical amino acid (NAA) technique. CuAAC is a popular reaction in which a terminal alkynyl group (HCC≡C—R₁) and an azido group (⁻N═N⁺═N—R₂) are united to give a 1,4-di-substituted 1,2,3-triazole in the presence of catalytic copper (See FIG. 1). One of ordinary skill in the art will appreciate that the R1 and R2 groups can vary and can include multiple types of substituents. Its uniqueness lies in the bio-orthogonality of both moieties, since they are absent in all natural amino acids and thus ensure a highly selective reaction. To employ CuAAC in protein engineering, amino acids containing either an alkynyl or azido group should be introduced into a protein. Among several techniques for expanding the chemical diversity of proteins (28-32), the site-specific genetic incorporation of NAAs is capable of adding new chemistries at a desired site.

In one embodiment, an orthogonal pair of tRNA amber suppressor and aminoacyl-tRNA synthetase from foreign species needs to be engineered to be specific for each NAA and utilized to incorporate it in response to an amber codon in the target protein sequence. In order to achieve site-specific fatty acid-conjugation to a protein, p-ethynylphenylalanine (pEthF) was introduced to a model protein, superfolder (sf) green fluorescent protein (sfGFP), using the bacterial cells outfitted with the orthogonal pair of engineered yeast phenylalanyl-tRNA/phenylalanyl-tRNA synthetase.

sfGFP was chosen as a model protein because of favorable properties. First, its fluorescence is directly correlated to its folding. Therefore, perturbation of its folded structure upon fatty acid-conjugation can be estimated by measuring its fluorescence. Second, its spectral properties greatly facilitate quantitative analyses in vitro including HSA binding assay. Third, the family of green fluorescent protein variants is generally known to be non-toxic to animals facilitating pharmacokinetics testing in vivo. The sfGFP variant containing pEthF was coupled to a fatty acid derivative containing an azido group via CuAAC. Finally, using the fatty acid-conjugated sfGFP, it is disclosed herein that the site-specific fatty acid-conjugation to a protein enhances its binding to HSA in vitro and prolongs protein retention in blood when administered in vivo without any significant loss in its intrinsic folded structure and fluorescence.

The present application provides compositions and methods for preparing mutant proteins and peptides wherein the mutant sites are inserted as sites for binding to a fatty acid. In one aspect, the fatty acid is palmitic acid.

Site-specificity is a critical key advantage of this new technique over other albumin-binding strategies relying on the genetic fusion of affinity motifs or random chemical attachment of synthetic binding molecules. Another key to exploiting this technology is imparting albumin-binding capability to a protein with minimal perturbation of its native activity and stability.

Compositions and methods for increasing the in vivo half-life of a protein or peptide are provided. In one aspect, the increase in half-life is in the blood. In one aspect, the increase in vivo half-life is in a human subject. In one aspect, the protein or peptide of interest is conjugated to a fatty acid. In one aspect, the protein-fatty acid conjugate is then conjugated to, or bound to, human serum albumin or other serum protein before being administered to a subject in need thereof.

Therapeutics proteins and peptides can include known proteins and peptides, and biologically active homologs and fragments thereof, useful for treating injuries, diseases and disorders. These can include cardiovascular disease, diabetes, trauma, wounds, cancer, endocrine and blood disorders, etc.

The therapeutic proteins and peptides can include, but are not limited to, cytokines, interferons, interleukins, lymphokines, and growth factors.

In one aspect, proteins and growth factors useful in the practice of the invention include, but are not limited to, EGF, PDGF, GCSF, IL6, IL8, IL10, MCP1, MCP2, Tissue Factor, FGFb, KGF, VEGF, PDGF, MMP1, MMP9, TIMP1, TIMP2, TGFβ, interferons, TNF-α, and HGF. One of ordinary skill in the art will appreciate that the choice of growth factor, cytokine, hormone, or extracellular matrix protein used will vary depending on criteria such as the age, health, sex, and weight of the subject, etc. In one aspect, the growth factors, cytokines, hormones, and extracellular matrix compounds and proteins are human.

Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha -beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, kit-ligand or FLT-3, angiostatin, thrombospondin, endostatin, neurotrophin, complement proteins, and LT.

As used herein, the term protein or peptide includes proteins and peptides from natural sources or from recombinant cell culture or synthetic molecules, as well as biologically fragments and homologs thereof. One of ordinary skill in the art will appreciate that the methods of the invention as disclosed herein can be used when, for example, certain NAAs are used to replace amino acids of a protein or peptide.

In one aspect, a fatty acid conjugated protein of the invention has an increased half-life in vivo relative to the wild type protein. In one aspect, a fatty acid conjugated protein conjugated to albumin or another serum protein has a longer half-life than the same protein not conjugated to a fatty-acid.

In one aspect, a modified protein or modified peptide of the invention has an increased half-life in vivo relative to the parent unmodified protein. In one aspect, the modification is a substitute NAA and conjugation to a fatty acid. In one aspect, the conjugated fatty acid-protein/peptide is also conjugated to or allowed to bind to another protein such as albumin. In one aspect, the half-life increases by at least about 10%. In another aspect, the half-life increases by at least about 20%. In another aspect, the half-life increases by at least about 50%. In another aspect, the half-life increases by at least about 100%. In another aspect, the half-life increases by at least about 200%. In another aspect, the half-life increases by at least about 500%. In another aspect, the half-life increases by at least about 1,000%. In another aspect, the half-life increases by at least about 5,000%. In another aspect, the half-life increases by at least about 10,000%. In another aspect, the half-life increases by at least about 20,000%. In another aspect, the half-life increases by at least about 50,000%. In another aspect, the half-life increases by at least about 100,000%. In one aspect, the increase in in vivo half-life is in a tissue. In one aspect, the increase in in vivo half-life is in the circulation (blood).

In one aspect, a modified protein or peptide of the invention can be labelled and used as a diagnostic.

When a modified protein or peptide of the invention is administered to a subject in need thereof, an effective amount can be administered by any suitable route or any suitable location on the subject. In one aspect, it is administered intravascularly. It can also be administered more than once. One of ordinary skill in the art can determine how much modified protein to administer, how many doses to administer, etc.

Various aspects and embodiments of the invention are described in further detail below.

### BRIEF DESCRIPTION OF THE DRAWINGS

FIG. 1. (FIG. 1A) Copper-catalyzed alkyne-azide cycloaddition. Chemical structures of p-ethynylphenylalanine (FIG. 1B) and palmitic acid-azide (FIG. 1C).

FIG. 2. (FIG. 2A) Incorporation of pEthF and fatty acid-conjugation at the 38^{th} position of the mDHFR-pEthF confirmed by MALDI-TOF analysis. Peptide F38 (top) of the mDHFR-WT and Peptide Z38 of the mDHFR-pEthF (bottom). (FIG. 2B) Protein gel images of the fluorogenic dye-treated mDHFR-pEthF (pEthF) and mDHFR-WT (WT). The gel was subjected to UV (360 nm) irradiation to excite the fluorophore (Fluorescence panel), and then stained with Coomassie brilliant blue (Coomassie panel) to visualize proteins.

FIG. 3. The relative fluorescence of the sfGFP-WT and sfGFP variants. Protein solutions (20 μg/mL) were loaded onto a 96-well microplate at 100 μL per well, and read on the plate reader at λₑₓ=480 nm and λₑₘ=510 nm. Values were averaged for each protein (n=5), and normalized to the fluorescence of the sfGFP-WT. In order to investigate the effect of reagents used in CuAAC, the sfGFP-WT was treated in parallel with the sfGFP-pEthF subjected to the fatty acid-conjugation, and designated sfGFP-WT (R).

FIG. 4. Relative albumin-binding affinities of sfGFP-variants. (FIG. 4A) Inactivated (amine-reactive functional groups blocked by glycine) or HSA-immobilized agarose beads were mixed with the sfGFP-WT and the sfGFP-Pal. After washing extensively with PBS, the fluorescence image was taken on the UV epi-illuminator at λₑₓ=480 nm, and emitted light above 510 nm was captured. For a quantitative fluorescence measurement, the same amounts of agarose beads were loaded on a 96-well microplate and read on the plate reader at λₑₓ=480 nm and λₑₘ=510 nm. The relative amounts of sfGFP samples were calculated from the relative fluorescence intensities. (FIG. 4B) Four micrograms of each protein in 2 μL of PBS were dotted onto the HSA-coated nitrocellulose membrane and air-dried. After washing in PBS for 5 min and air-dry, the membrane was epi-illuminated at λₑₓ=480 nm, and emitted light above 510 nm was captured.

FIG. 5. Pharmacokinetics of the sfGFP-WT and the sfGFP-Pal. Four mice were intravenously administered the sfGFP-Pal (square) or the sfGFP-WT (triangle), respectively, and serum concentrations were measured by ELISA at different time points: 0 (10 min), 3, and 6 hr for the sfGFP-WT; 0 (10 min), 3, 6, 24, and 30 hr for the sfGFP-Pal. Data were normalized with regard to the initial value, plotted in a logarithmic scale versus time post-injection, and fitted into a straight line (R²=0.98 for the sfGFP-WT and 0.97 for the sfGFP-Pal).

FIG. 6. Liquid chromatography tandem MS of Peptide Z38 (m/z=1706.8, NGDLPWPPLRNEAZK) of the mDHFR-pEthF (FIG. 2). The system consisted of a Thermo Electron LTQ Orbitrap XL mass spectrometer interfaced to a Phenomenex Jupiter C18. The analysis was performed by acquiring a mass spectrum using Fourier transform ion cyclotron resonance.

FIG. 7. Fatty acid-conjugation at the 38^{th} position of the mDHFR-pEthF confirmed by MALDI-TOF MS analysis (Peptide Z38-PAL of the mDHFR-Pal).

FIG. 8. (FIG. 8A) Residue-based solvent accessibility of the sfGFP-pEthF. ASAView (3), an online tool for a graphical representation of solvent accessibility, was used to calculate the relative solvent accessibility of all residues including pEthF at 215^{th} position, based on the PDB file (ID: 2B3P) of sfGFP and a fully automated protein structure homology-modeling server (4) for mutation. (FIG. 8B) Three-dimensional structure of the sfGFP-pEthF generated by Pymol (5). The chromophore (orange) and pEthF incorporated at the 215^{th} position (magenta) are represented by spheres.

FIG. 9. ESI-MS spectra of the sfGFP-pEthF (FIG. 9A) and the sfGFP-Pal (FIG. 9B). Following the reversed-phase high performance chromatography using BEH C4 column (2.1×100 mm, 1.7 μm), the molecular weight of a full length protein was analyzed on an LTQ-Orbitrap XL mass spectrometer.

### DETAILED DESCRIPTION

Abbreviations and Acronyms

AFWK—a Phe/Trp/Lys triple auxotrophic E. coli strain

CuAAC—copper-catalyzed alkyne-azide cycloaddition

DHFR—dihydrofolate reductase

ESI—electron spray ionization

HSA—human serum albumin

m—murine

mDHFR—murine dihydrofolate reductase

MS—mass spectrometry

NAA—noncanonical amino acid

p—para position

Pal—palmitic acid-azide

pEthF—p-ethynylphenylalanine

sf—superfolder

sfGFP—superfolder green fluorescent protein

TFA—trifluoroacetic acid

TGF—transforming growth factor

WT—wild type

Definitions

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” refers to a substance that elicits an enhanced immune response when used in combination with a specific antigen.

As use herein, the terms “administration of” and or “administering” a compound should be understood to mean providing a compound of the invention or a prodrug of a compound of the invention to a subject in need of treatment.

As used herein, the term “aerosol” refers to suspension in the air. In particular, aerosol refers to the particlization or atomization of a formulation of the invention and its suspension in the air.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

As used herein, “albumin,” in the context of “human serum albumin,” has a long serum half-life and can function as a blood transport carrier for molecules, such as those of low water solubility, including but not limited to, lipid soluble hormones, bile salts, free fatty acids, and drugs. It has low binding affinity for many types of molecules.

“Albumin-binding affinity,” as used herein, refers to the interaction of a ligand binding to human serum albumin protein. High-affinity ligand binding results from greater intermolecular force between the ligand and albumin, and it involves a longer residence time for the ligand at its receptor binding site. Low-affinity ligand binding involves less intermolecular force between the ligand and albumin, and it involves a shorter residence time for the ligand at its receptor binding site.

The term “alkyne” or “alkyne group,” as used herein, refers to an unsaturated hydrocarbon containing at least one carbon-carbon triple bond between two carbon atoms.

An “antagonist” is a composition of matter which when administered to a mammal such as a human, inhibits a biological activity attributable to the level or presence of a compound or molecule of interest in the mammal.

As used herein, “alleviating a disease or disorder symptom,” means reducing the severity of the symptom or the frequency with which such a symptom is experienced by a patient, or both.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. “Nonstandard amino acids” include “non-natural” and “noncanonical” amino acids. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

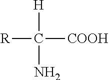

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a subject, including, but not limited to, skin, hair, tissue, blood, plasma, cells, sweat and urine.

The term “cancer”, as used herein, is defined as proliferation of cells whose unique trait—loss of normal controls—results in unregulated growth, lack of differentiation, local tissue invasion, and metastasis. Examples include but are not limited to, melanoma, breast cancer, prostate cancer, ovarian cancer, uterine cancer, cervical cancer, skin cancer, pancreatic cancer, colorectal cancer, renal cancer and lung cancer.

As used herein, the term “carrier molecule” refers to any molecule that is chemically conjugated to the antigen of interest that enables an immune response resulting in antibodies specific to the native antigen.

The term “cell surface protein” means a protein found where at least part of the protein is exposed at the outer aspect of the cell membrane. Examples include growth factor receptors.

As used herein, the term “chemically conjugated,” or “conjugating chemically” refers to linking a chemical or peptide/protein to another protein.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

A “conjugated protein” refers to a protein containing one or more prosthetic groups.

As used herein, “conjugation” refers to the process of linking a molecule or substance, such as a fatty acid chain or therapeutic protein, to another molecule, such as a protein or carrier molecule, via p-orbital overlap. This linking can occur on the genetic level using recombinant technology, wherein a hybrid protein may be produced containing the amino acid sequences, or portions thereof, of both an antigen and carrier molecule. This hybrid protein is produced by an oligonucleotide sequence encoding both the antigen and the carrier molecule, or portions thereof. This linking can also occur via chemical reaction, including but not limited to, copper-catalyzed alkyne-azide cycloaddition.

The term “cycloaddition,” in the context of “copper-catalyzed alkyne-azide cycloaddition,” refers to an organic reaction catalyzed by copper in which an organic azide group reacts neatly with a terminal alkyne group to produce a triazole. The use of this chemical reaction includes, but is not limited to, the coupling of polymers with other polymers or small molecules. As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

As used herein, the term “effector domain” refers to a domain capable of directly interacting with an effector molecule, chemical, or structure in the cytoplasm which is capable of regulating a biochemical pathway.

The term “elixir,” as used herein, refers in general to a clear, sweetened, alcohol-containing, usually hydroalcoholic liquid containing flavoring substances and sometimes active medicinal agents.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

The term “growth factor” as used herein means a bioactive molecule that promotes the proliferation of a cell or tissue. Growth factors useful in the present invention include, but are not limited to, transforming growth factor-alpha (TGF-α), transforming growth factor-beta (TGF-β), platelet-derived growth factors including the AA, AB and BB isoforms (PDGF), fibroblast growth factors (FGF), including FGF acidic isoforms 1 and 2, FGF basic form 2, and FGF 4, 8, 9 and 10, nerve growth factors (NGF) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors may also promote differentiation of a cell or tissue. TGF, for example, may promote growth and/or differentiation of a cell or tissue.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

By the term “immunizing a subject against an antigen” is meant administering to the subject a composition, a protein complex, a DNA encoding a protein complex, an antibody or a DNA encoding an antibody, which elicits an immune response in the subject, and, for example, provides protection to the subject against a disease caused by the antigen or which prevents the function of the antigen.

The term “immunologically active fragments thereof” will generally be understood in the art to refer to a fragment of a polypeptide antigen comprising at least an epitope, which means that the fragment at least comprises 4 contiguous amino acids from the sequence of the polypeptide antigen.

As used herein the term “the modified protein has increased binding affinity for a serum protein” means that it has increased activity relative to its parent unmodified protein.

As used herein, the term “induction of apoptosis” means a process by which a cell is affected in such a way that it begins the process of programmed cell death, which is characterized by the fragmentation of the cell into membrane-bound particles that are subsequently eliminated by the process of phagocytosis.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

The term “inhibit a complex,” as used herein, refers to inhibiting the formation of a complex or interaction of two or more proteins, as well as inhibiting the function or activity of the complex. The term also encompasses disrupting a formed complex.

However, the term does not imply that each and every one of these functions must be inhibited at the same time.

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

The term “injury” refers to any physical damage to the body caused by violence, accident, trauma, or fracture, etc., as well as damage by surgery.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target compound or molecule.

A “receptor” is a compound that specifically binds to a ligand.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The terms “modified protein” or “modified peptide” as used herein refers to a protein or peptide in which a nonstandard amino acid has been added to the parent protein (unmodified) or peptide or has a substituted amino acid in the protein or peptide, and a fatty acid has been conjugated to the incorporated nonstandard amino acid.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

As used herein, “p-ethynylphenylalanine” is a non-natural amino acid comprising a phenylalanine analog with an alkyne moiety at para-position of the phenyl ring.

The term “peptide” typically refers to short polypeptides.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “population of cells” as used herein refers to a mixed population such as blood, bone marrow-derived, or umbilical cord blood cells. By the term “at least two different populations of cells” is meant the original sources are different, such as obtaining two or more different lots/units of umbilical cord blood, or umbilical cord blood from a source combined with bone marrow-derived cells from another source, etc. In some instances, the “population of cells” can be subjected to methods for enriching a cell type, such as CD133 or CD34 cells. Of course, if methods are found to obtain pure populations of CD133 or CD34 cells, these cells are encompassed by the methods of the invention as well.

By “presensitization” is meant pre-administration of at least one innate immune system stimulator prior to challenge with an agent. This is sometimes referred to as induction of tolerance.

The term “prevent,” as used herein, means to stop something from happening, or taking advance measures against something possible or probable from happening. In the context of medicine, “prevention” generally refers to action taken to decrease the chance of getting a disease or condition. A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of developing pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “receptor” is a compound that specifically binds to a ligand.

A “ligand” is a compound that specifically binds to a target receptor.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., eds., 1994, Methods for General and Molecular Bacteriology, American Society for Microbiology, Washington, D.C., p. 574).

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, “serum” refers to the protein-rich liquid remaining after blood has clotted.

The term “serum half-life” is the term used when describing the amount of time a molecule exists when administered into the blood of a subject and is not limited to “serum”.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

As used herein, “site-specific” refers to incorporation of a certain molecule into another molecule or conjugation of a certain molecule to another molecule at a specific position of the second molecule.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this invention.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2×standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

By the term “vaccine,” as used herein, is meant a composition which when inoculated into a subject has the effect of stimulating an immune response in the subject, which serves to fully or partially protect the subject against a condition, disease or its symptoms. In one aspect, the condition is conception. The term vaccine encompasses prophylactic as well as therapeutic vaccines. A combination vaccine is one which combines two or more vaccines, or two or more compounds or agents.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

As used herein, the term “wound” relates to a physical tear, break, or rupture to a tissue or cell layer. A wound may occur by any physical insult, including a surgical procedure or as a result of a disease, disorder condition.

### Embodiments

Depending on the protein or peptide to be modified by inserting site-specific NAA for conjugating with a fatty acid the present application discloses new methods for modification, but other methods can be used to modify proteins as well when needed.

In one embodiment, the invention provides a method for incorporating an amino acid into a protein. In one aspect, the amino acid is a non-natural amino acid. In yet a further aspect, the non-natural amino acid is p-ethynylphenylalanine. In another aspect, the non-natural amino acids that are useful with the practice of the invention include, but are not limited to, dehydralanine, carboxyglutamic acid, selenocysteine, pyrrolysine, N-formylmethionine, or any other non-natural amino acid. The present invention provides for incorporation of one or more of the amino acids in the natural L-isomeric form or the D-isomeric form.

One embodiment of the present invention provides compositions and methods for increasing protein half-life. In one aspect, the present invention provides compositions and methods for increasing in vivo protein half-life. In one aspect, it is in blood. In another aspect, it is in serum. In another aspect, the present invention provides compositions and methods for increasing in vitro protein half-life. The present invention provides for conjugation of a free fatty acid to an amino acid incorporated into a protein. In one aspect, the fatty acid is palmitic acid. In another aspect, the fatty acid includes, but is not limited to, pentadecylic acid, margaric acid, stearic acid, nonadecylic acid, arachidic acid, heneicosylic acid, behenic acid, tricosylic acid, lignoceric acid, pentacosylic acid, or any other saturated fatty acid. In yet another aspect, the fatty acid includes, but is not limited to, linoleic acid, arachidonic acid, stearidonic acid, palmitoleic acid, vaccenic acid, paullinic acid, oleic acid, or any other unsaturated fatty acid.

In one embodiment, the present invention provides for conjugation of a fatty acid to a protein via chemical cycloaddition. In one aspect, this chemical cycloaddition comprises copper-catalyzed alkyne-azide cycloaddition. In another aspect, the cycloaddition includes, but is not limited to, transition metal-catalyzed or mediated [5+1] cycloadditions, formal [3+3] cycloaddition, and cycloreversion.

In one embodiment, the present invention provides for a method of increasing protein half-life by conjugating a therapeutic protein to a fatty acid, which binds to a fatty-acid-binding protein . In one aspect, this fatty-acid-binding protein is albumin. In yet a further aspect, this albumin is human serum albumin. In another aspect, this fatty-acid-binding protein includes, but is not limited to, FABP 1, FABP 2, FABP 3, FAPB 4, FABP 5, FABP 6, FABP 7, FABP 8, FABP 9, FABP 11, FABP 12, FABP 5-like 1, FABP 5-like 2, FABP 5-like 3, FABP 5-like 4, FABP 5-like 5, FABP 5-like 6, FABP 5-like 7, or any other protein that is able to bind fatty acids. In one aspect, this fatty-acid-binding protein has a low clearance rate. In one aspect, the fatty-acid-binding protein is a serum protein.

Other useful proteins of the invention for modification include growth factors, cytokines, etc., as well as therapeutic proteins and proteins used as carriers or for imaging purposes, such as labeled proteins. The term “growth factor” as used herein means a bioactive molecule that promotes the proliferation of a cell or tissue. Growth factors useful in the present invention include, but are not limited to, transforming growth factor-alpha (TGF-α), transforming growth factor-beta (TGF-β), platelet-derived growth factors including the AA, AB and BB isoforms (PDGF), fibroblast growth factors (FGF), including FGF acidic isoforms 1 and 2, FGF basic form 2, and FGF 4, 8, 9 and 10, nerve growth factors (NGF) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors may also promote differentiation of a cell or tissue. TGF, for example, may promote growth and/or differentiation of a cell or tissue. Note that many factors are pleiotropic in their activity and the activity can vary depending on things such as the cell type being contacted, the state of proliferation or differentiation of the cell, etc. Additional growth factors and cytokines include, but are not limited to, PDGF, FGF, TNFα, IL-6, and endothelin-1.

The present invention encompasses the use of the proteins described herein as well as biologically active homologs and fragments thereof. The proteins are known in the art. NCBI GenBank Accession numbers for the human proteins described herein, including some precursors, fragments, and isoforms are provided below.

TGFβ—Accession: AAA36738.1, 431 aa.

Insulin-like growth factor-binding protein 2 (IGFBP-2)- Accession: NP_{—}000588.2, 328 aa.

IGFBP-3—The encoded protein includes a 27-residue signal peptide followed by the 264-residue mature protein. IGFBP-3 shares with the other five high-affinity IGFBPs and a 3-domain structure:
isoform a precursor, 297 aa protein, Accession: NP_{—}001013416.1
isoform b precursor, 291 aa protein, Accession: NP_{—}000589.2 and Accession: P17936.2

Monocyte Chemoattractant Protein-1 (also referred to as CCL2). Mature human MCP-1 is composed of 76 amino acids and is 13 kDa in size. The precursor also has a 23 amino acid signal peptide; Accession: AAB20651.1, 99 aa

Osteopontin—

300 aa protein, Accession: AAA59974.1 or AAC28619.1 or NP_{—}000573.1

314 aa protein, Accession: AAA86886.1 or BAA03554.1 or P10451.1 or NP_{—}001035147.1

273 aa protein, Accession: BAH58215.1 or BAE45628.1 or NP_{—}001238758.1

287 aa protein, Accession: NP_{—}001035149.1

isoform 5, 327 aa protein, Accession: NP_{—}001238759.1

SDF-1—Processed forms SDF-1-beta (3-72) and SDF-1-alpha (3-67) are produced after secretion by proteolytic cleavage of isoforms Beta and Alpha, respectively.

93 aa protein, Accession: P48061.1

VEGF—There are multiple isoforms of VEGFA that result from alternative splicing of mRNA from a single, 8-exon VEGFA gene. These are classified into two groups which are referred to according to their terminal exon (exon 8) splice site: the proximal splice site (denoted VEGFxxx) or distal splice site (VEGFxxxb). In addition, alternate splicing of exon 6 and 7 alters their heparin-binding affinity and amino acid number (in humans: VEGF121, VEGF121b, VEGF145, VEGF165, VEGF165b, VEGF189, VEGF206). VEGFA has been classically described as having four main splice variants (121, 165, 189, and 206), although other splice variants have been described as present. VEGF165b, a form of VEGF165, is a splice variant on which exon 8 has a 6-amino acid difference from the typical VEGF165. VEGF165b is an antiangiogenic VEGFA isoform.

splice variant VEGF 117, 143 aa protein, Accession: AAP86646.1

precursor, 232 aa protein, Accession: P15692.2

isoform s, 163 aa protein, Accession: NP_{—}001273973.1

isoform g, 371 aa protein, Accession: NP_{—}001028928.1

isoform e, 354 aa protein, Accession: NP_{—}001020540.2

isoform d, 371 aa protein, Accession: NP_{—}001020539.2

isoform c, 389 aa protein, Accession: NP_{—}001020538.2

isoform b, 395 aa protein, Accession: NP_{—}003367.4

isoform a, 412 aa protein, Accession: NP_{—}001020537.2

isoform o precursor, 191 aa protein, Accession: NP_{—}001165100.1

isoform m precursor, 174 aa protein, Accession: NP_{—}001165098.1

isoform l precursor, 191 aa protein, Accession: NP_{—}001165097.1

isoform k precursor, 209 aa protein, Accession: NP_{—}001165096.1

isoform j precursor, 215 aa protein, Accession: NP_{—}001165095.1

isoform i precursor, 232 aa protein, Accession: NP_{—}001165094.1

isoform h, 317 aa protein, Accession: NP_{—}001165093.1

isoform f, 327 aa protein, Accession: NP_{—}001020541.2

isoform p precursor, 137 aa protein, Accession: NP_{—}001165101.1

isoform n precursor, 147 aa protein, Accession: NP_{—}001165099.1

PDGF is a dimeric glycoprotein composed of two A (-AA) or two B (-BB) chains or a combination of the two (-AB).

PDGF-A

isoform 2 preproprotein, 196 aa protein, Accession: NP_{—}148983.1

isoform 1 preproprotein, 211 aa protein, Accession: NP_{—}002598.4

234 aa protein, Accession: AAI09247.1

PDGFB

241 aa protein, Accession: 1109245A

isoform 2 preproprotein, 226 aa protein, Accession: NP_{—}148937.1

FGF—The FGFs are heparin-binding proteins and interactions with cell-surface-associated heparan sulfate proteoglycans have been shown to be essential for FGF signal transduction. FGF1 is also known as acidic, and FGF2 is also known as basic fibroblast growth factor. One important function of FGF1 and FGF2 is the promotion of endothelial cell proliferation and the physical organization of endothelial cells into tube-like structures. They thus promote angiogenesis, the growth of new blood vessels from the pre-existing vasculature. FGF1 and FGF2 are more potent angiogenic factors than vascular endothelial growth factor (VEGF) or platelet-derived growth factor (PDGF). When the term FGF is used herein, it encompasses the use of FGF1 or FGF2 as well as biologically active homologs and fragments thereof.

FGF1 isoform 1 precursor, 155 aa protein, Accession: NP_{—}001244139.1 or AAH32697.1 or P05230.1

FGF2, 288 aa protein, Accession: NP_{—}001997.5

TNFα—233 aa protein, Accession: NP_{—}000585.2 or AAA61200.1.

Interleukin-6

precursor, 212 aa protein, Accession: NP_{—}000591.1 or AAD13886.1.

211 aa protein, Accession: AFF18412.1

185 aa protein, Accession: AAB30962.1

Endothelin-1

212 aa protein, Accession: P05305.1 or AAA52339.1

partial, 51 aa protein, Accession: AAA52341.1.

In one embodiment, the invention provides for a method of increasing super-folder green fluorescent protein (sfGFP) in vivo half-life at least about 1 hour. In another embodiment, the invention provides for a method of increasing sfGFP in vivo half-life at least about 2 hours. In another embodiment, the invention provides for a method of increasing sfGFP in vivo half-life at least about 3 hours. In another embodiment, the invention provides for a method of increasing sfGFP in vivo half-life at least about 4 hours. In another embodiment, the invention provides for a method of increasing sfGFP in vivo half-life at least about 5 hours.

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters. Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide. Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4 -, C8- or C18- silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation,” a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones, or amides. Ester or ketone-forming alkyl groups, particularly lower alkyl groups such as methyl, ethyl and propyl, and amide-forming amino groups such as primary amines (—NH₂), and mono-and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

The present invention also provides for homologs of proteins and peptides. Homologs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on protein function.

Modifications (which do not normally alter primary sequence) include in vivo, or in vitro chemical derivatization of polypeptides, e.g., acetylation, or carboxylation.

Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Homologs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substantially pure protein or peptide obtained as described herein may be purified by following known procedures for protein purification, wherein an immunological, enzymatic, or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

### Amino Acid Substitutions

In addition to site specific modifications with NAA as disclosed herein, the proteins and peptides of the invention can be modified in other ways if needed. In certain embodiments, the disclosed methods and compositions may involve preparing peptides with one or more substituted amino acid residues. In various embodiments, the structural, physical and/or therapeutic characteristics of peptide sequences may be optimized by replacing one or more amino acid residues.

In one embodiment, the invention encompasses the substitution of a serine or an alanine residue for a cysteine residue in a peptide of the invention. Support for this includes what is known in the art. For example, see the following citation for justification of such a serine or alanine substitution: Kittlesen et al., 1998 Human melanoma patients recognize an HLA-A1-restricted CTL epitope from tyrosinase containing two cysteine residues: implications for tumor vaccine development J Immunol., 60, 2099-2106.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art. For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma'-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (—2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys

(C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) tip, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Tip. (See, e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

The invention is also directed to methods of administering useful compounds or cells with the modified proteins and peptides (collectively referred to as compounds) of the invention to a subject.

Pharmaceutical compositions comprising the present compounds are administered to an individual in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

The present invention is also directed to pharmaceutical compositions comprising the peptides of the present invention. More particularly, such compounds can be formulated as pharmaceutical compositions using standard pharmaceutically acceptable carriers, fillers, solublizing agents and stabilizers known to those skilled in the art.

The invention also encompasses the use pharmaceutical compositions of an appropriate compound, homolog, fragment, analog, or derivative thereof to practice the methods of the invention, the composition comprising at least one appropriate compound, homolog, fragment, analog, or derivative thereof and a pharmaceutically-acceptable carrier.

The pharmaceutical compositions useful for practicing the invention may be administered to deliver a dose of between 1 ng/kg/day and 100 mg/kg/day.

Pharmaceutical compositions that are useful in the methods of the invention may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the invention.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of biotechnology and pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

According to an embodiment, a formulation of the invention contains an antimicrobial agent. The antimicrobial agent may be provided at, for example, a standard therapeutically effective amount. A standard therapeutically effective amount is an amount that is typically used by one of ordinary skill in the art or an amount approved by a regulatory agency (e.g., the FDA or its European counterpart).

The composition of the invention can further comprise additional therapeutic agents, alone or in combination (e.g., 2, 3, or 4 additional additives). Examples of additional agents include but are not limited to: (a) antimicrobials, (b) steroids (e.g., hydrocortisone, triamcinolone); (c) pain medications (e.g., aspirin, an NSAID, and a local anesthetic); (d) anti-inflammatory agents; and (e) combinations thereof.

In other embodiments, therapeutic agents, including, but not limited to, cytotoxic agents, anti-angiogenic agents, pro-apoptotic agents, antibiotics, hormones, hormone antagonists, chemokines, drugs, prodrugs, toxins, enzymes, or other agents may be used. Additionally, they may be used as adjunct therapies when using the liposome complexes described herein. Drugs useful in the invention may, for example, possess a pharmaceutical property selected from the group consisting of antimitotic, antikinase, alkylating, antimetabolite, antibiotic, alkaloid, anti-angiogenic, pro-apoptotic agents, and combinations thereof. In one aspect, the drug or agent is encapsulated into a liposome of the invention.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Examples of antimicrobial agents that can be used in the present invention include, but are not limited to, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, cikprofloxacin, doxycycline, ampicillin, amphotericine B, ketoconazole, fluconazole, pyrimethamine, sulfadiazine, clindamycin, lincomycin, pentamidine, atovaquone, paromomycin, diclarazaril, acyclovir, trifluorouridine, foscarnet, penicillin, gentamicin, ganciclovir, iatroconazole, miconazole, Zn-pyrithione, and silver salts, such as chloride, bromide, iodide, and periodate.

The invention also includes a kit comprising the composition of the invention and an instructional material which describes administering or using the composition. In another embodiment, this kit comprises a (preferably sterile) solvent suitable for dissolving or suspending the composition of the invention prior to administering the composition. Optionally, at least one growth factor and/or antimicrobial agent may be included in the kit.

Without further description, it is believed that one of ordinary skill in the art can, using the preceding description and the following illustrative examples, make and utilize the compounds of the present invention and practice the claimed methods. The following examples therefore, specifically point out the preferred embodiments of the present invention, and are not to be construed as limiting in any way the remainder of the disclosure.

### Examples

### Materials

p-ethynylphenylalanine (pEthF) was synthesized as described previously (1). Ni-NTA agarose and pQE-16 plasmid were obtained from Qiagen (Valencia, Calif.). Sequencing grade modified trypsin was obtained from Promega (Madison, Wis.). Amicon ultra centrifugal filters and ZipTip® with C₁₈ media were purchased from Millipore (Billerica, Mass.). NHS-Activated Agarose and BCA Protein Assay Kit were purchased from Thermo Scientific (Rockford, Ill.). GFP ELISA kit was purchased from Cell Biolab, Inc. (San Diego, Calif.). Coumarin-azide was obtained from Glen Research (Sterling, Va.). Palmitic acid-azide was obtained from Invitrogen (Carlsbad, Calif.). All other chemicals were purchased from Sigma (St. Louis, Mo.).

### Plasmids Construction and Strains

Preparation of the plasmids pQE16am-yPheRS^{T415G} and pREP4-ytRNA^{Phe}_{CUA}_{_{—}}_{UG} is described previously (2). pQE16am-yPheRS^{T415G} encodes the engineered yeast aminoacyl-tRNA synthetase and the murine dihydrofolate reductase (mDHFR) with an amber codon at 38^{th} position and a C-terminal hexahistidine tag. A Phe/Trp/Lys triple auxotrophic E. coli strain, designated AFWK, was prepared as described previously (2). AFWK harboring both plasmids was used as an expression host for incorporation of pEthF into murine dihydrofolate reductase (mDHFR). A gene encoding a sfGFP with a C-terminal hexahistidine tag was synthesized from Epoch Life Science (Sugar Land, Tex.). The expression cassette of the sfGFP was inserted into the AatI/NheI site in pQE16am-yPheRS^{T415G} replacing the coding sequence of the mDHFR and yielding pQE16-sfGFP-yPheRS^{T415G}. An amber codon was generated by PCR mutagenesis in a position between the 214^{th} and the 215^{th} amino acid of the sfGFP resulting in pQE16-sfGFP_{215Amb}-yPheRS^{T415G}. The mutagenic primer sequences were as follows: 215Amb_F, 5′-CCCAACGAAAAGTAGCGTGACCACATGG-3′ (SEQ ID No: 1); 215Amb_R, 5′-CCATGTGGTCACGCTACTTTTCGTTGGG-3′ (SEQ ID No: 2). AFWK was co-transformed with pQE16-sfGFP_{215Amb}-yPheRS^{T415G} and pREP4-ytRNA^{Phe}_{CUA}_{_{—}}^{UG} and then used as an expression host for incorporation of pEthF into the sfGFP.

### Expression and Purification of Wild-Type and Mutant Proteins

The wild-type sfGFP (sfGFP-WT) was expressed from AFWK harboring pQE16-sfGFP-yPheRS^{T415G} by 1 mM IPTG induction in LB media containing 100 μg/mL ampicillin at 37° C. To express the sfGFP mutant containing pEthF at the 215^{th} position (sfGFP-pEthF), AFWK harboring pQE16-sfGFP_{215Amb}-yPheRS^{T415G} and pREP4-ytRNA^{Phe}_{CUA}_{_{—}}_{UG} was used. Saturated overnight cultures grown at 37° C. in M9 minimal medium supplemented with 100 μg/mL ampicillin, 30 μg/mL kanamycin, 0.4% (w/v) glucose, 1 mM MgSO₄, 0.1 mM CaCl₂, 10 μg/mL thiamine, and 20 amino acids (25 μg/mL each) were diluted 20 fold in the same fresh medium, and grown at 37° C. until an OD₆₀₀ of 0.9 was reached. After incubation on ice for 15 min, cells were sedimented by centrifugation at 4,000 g for 12 min, and washed with cold 0.9% (w/v) NaCl by gentle resuspension. After repeating twice, cells were shifted to M9 medium supplemented with the same ingredients described above except for different amino acids composition: 17 amino acids (35 μg/mL each), 150 μM Lys, 60 μM Phe, 10 μM Trp, and 3 mM pEthF. To maximize the incorporation efficiency in condensed culture, the total volume of M9 medium was 20-fold smaller than the original volume. Upon induction by 1 mM IPTG, cells were incubated with shaking at 30° C. for 15 h before harvest. Expression of the wild-type mDHFR (mDHFR-WT) or the mDHFR mutant with pEthF (mDHFR-pEthF) at the 38^{th} position was performed similarly except that the plasmid pQE16 for mDHFR-WT or pQE16am-yPheRS^{T415G} for mDHFR-pEthF was used instead of pQE16-sfGFP_{215Amb}-yPheRS^{T415G}. Cells were pelleted by centrifugation, and the protein was purified by gravity-flow affinity chromatography using Ni-NTA agarose beads under native (sfGFP-WT and sfGFP-pEthF) or denaturing (mDHFR-WT and mDHFR-pEthF) condition according to the supplier's instructions (Qiagen). Purified proteins were directly used or buffer-exchanged using PD-10 desalting columns to appropriate buffers. If necessary, the protein solutions were concentrated using centrifugal filters.

### CuAAC-Mediated Dye Labeling and Fatty Acid-Conjugation

Palmitic acid-azide was reacted with the mDHFR-pEthF under the following condition yielding the mDHFR-Pal: 30 μM mDHFR-pEthF in the denaturing elution buffer (8 M urea, 10 mM Tris, 100 mM NaH₂PO₄, pH=4.5), 150 μM palmitic acid-azide, 1 mM CuSO₄, 2 mM sodium ascorbate, and at room temperature for 2 hr. sfGFP-pEthF was conjugated to palmitic acid-azide or fluorogenic coumarin-azide under the following condition generating sfGFP-Pal or sfGFP-CM: 30 μM sfGFP-pEthF in 20 mM potassium phosphate (pH=8) and 35% (v/v) DMSO, 150 μM palmitic acid-azide or coumarin-azide, 1.0 mM TBTA, 1.5 mM CuSO₄, 2.0 mM DTT, and at 25° C. for 10 hr. Reactions were quenched by adding 200 mM imidazole and 5 mM EDTA. Upon completion of reaction, the reaction mixture was desalted and buffer-exchanged using PD-10 desalting columns to appropriate buffers for downstream uses. Protein concentrations were determined by BCA assay.

Verification of pEthF Incorporation and Fatty Acid-Conjugation by Mass Spectrometry

Tryptic digestion of the mDHFR-WT, the mDHFR-pEthF or the mDHFR-Pal in the denaturing elution buffer (8 M urea, 10 mM Tris, 100 mM NaH₂PO₄, pH=4.5) was performed by diluting 10 μL of a protein with 90 μL of NH₄HCO₃ and then adding 0.5 μL of modified trypsin (0.1 μg). Following incubation at 37° C. for 2 h, the reaction mixture was mixed with 12 μL of 5% (v/v) trifluoroacetic acid (TFA) to quench the reaction and then desalted on a ZipTip® C₁₈. The site-specific incorporation of pEthF into mDHFR and palmitic acid-conjugation was confirmed by MALDI-TOF mass spectrometry (MS) analysis of the tryptic digests of mDHFR. The MS analysis was performed using 20 mg/mL of 2,5-dihydroxybenzoic acid and 2 mg/mL of L-(−)-fucose dissolved in 10% ethanol as a matrix by Microflex™ MALDI-TOF MS (Bruker, Billerica, Mass.). LC-MS/MS analyses of tryptic digests of mDHFR were conducted on a Thermo Electron LTQ VelosOrbitrap Mass Spectrometer. The tryptic digests of mDHFR were separated on a reverse phase column (75 μm) with acetonitrile gradient. The column eluent was introduced to the microspray source, and amino acid sequence analysis was carried out by fragmentation of the precursor ion corresponding to the Peptide_Z38. The site-specific incorporation of pEthF into sfGFP and palmitic acid-conjugation were confirmed by LC-MS coupled with electron spray ionization (ESI). The chromatographic separation was performed using a BEH C4 (2.1×100 mm, 1.7 μm) column at a flow rate of 0.4 μL/min with mobile phase consisting of water and n-propanol. The eluent was introduced into the ion source of the LTQ-Orbitrap mass spectrometer operated in positive mode at a spray voltage of 3.0 kV. The data were acquired by Xcalibur (Thermo Scientific) and processed using ProMass deconvolution (Thermo Scientific).

### In Vitro Albumin-Binding Assay

N-hydroxysuccinimide-activated agarose was coated with HSA according to the supplier's protocol or inactivated by adding excess amount of glycines to generate HSA-coated and inactivated resin, respectively. The resins were mixed with the sfGFP-WT, the sfGFP-pEthF, or the sfGFP-Pal and incubated at room temperature for 1 hr. After washing with PBS multiple times, the fluorescence images and intensities of the resins were obtained at λₑₓ=480 nm and λₑₘ=510 nm using Biospectrum imaging system (UVP, Inc, Upland, Calif.) and Biotek fluorescence plate reader, respectively. For membrane-based binding assay, the HSA solution (10 mg/mL) was spotted on the nitrocellulose membrane. After extensive washing with PBS, the membrane was blocked with casein solution. Two microliters of protein solutions (2 mg/mL) was overlaid on the HSA spot, and the membrane was washed with PBS and analyzed by the imaging system.

In Vivo Studies of the sfGFP-WT and sfGFP-Pal

The animal protocol was approved by the Institutional Animal Care and Use Committee (IACUC) at the University of Virginia. Pharmacokinetic properties of sfGFP-WT and sfGFP-Pal were investigated by injecting 50 μg of each sfGFP sample in 200 μL PBS into the tail vein of young female C57BL/6 mice (n=4). The blood was sampled at 0 (10 min), 3, and 6 hr post-injection for the sfGFP-WT, and at 0 (10 min), 3, 6, 18, 24, and 30 hr post-injection for the sfGFP-Pal.

### Results

Site-Specific Incorporation of p-ethynylphenylalanine into the Murine Dihydrofolate Reductase and the CuAAC-Mediated Dye Labeling

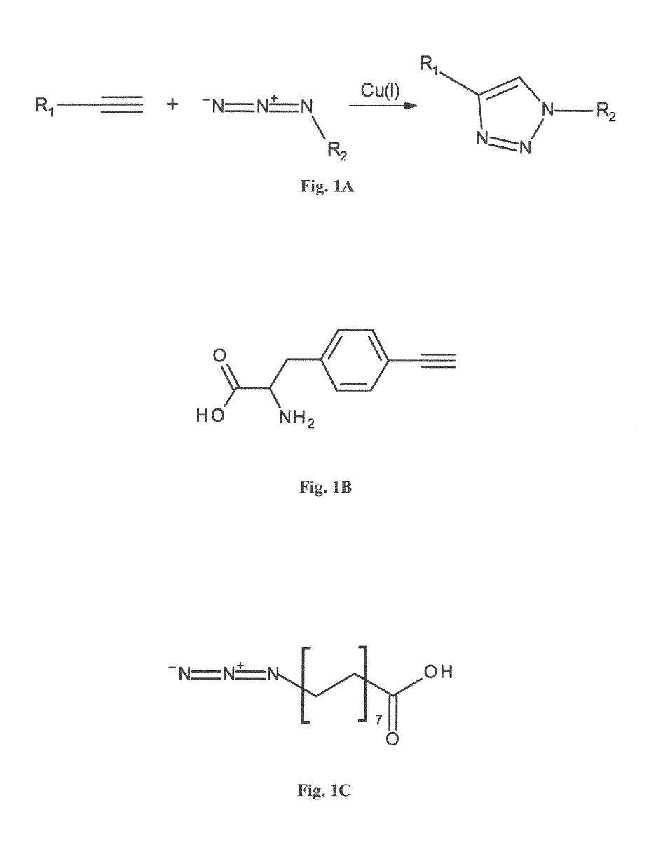

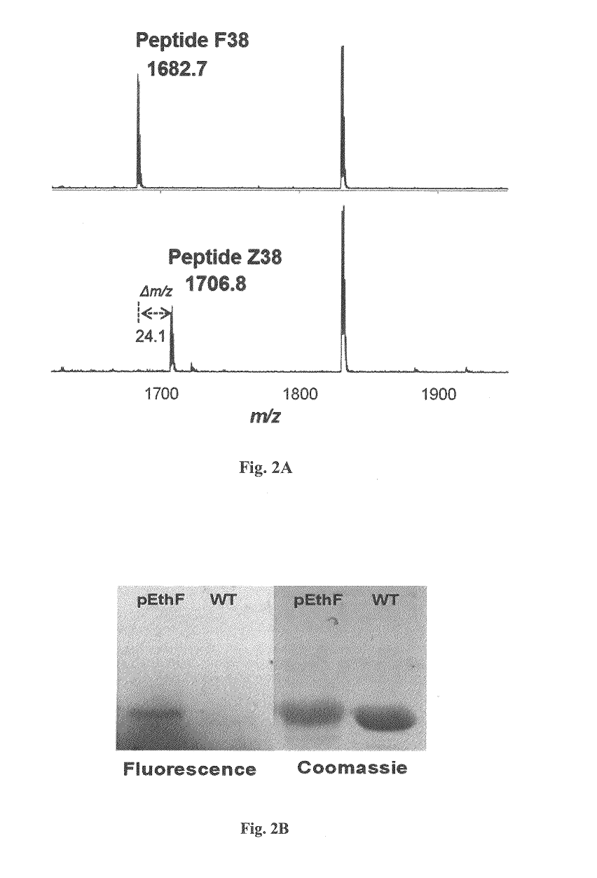

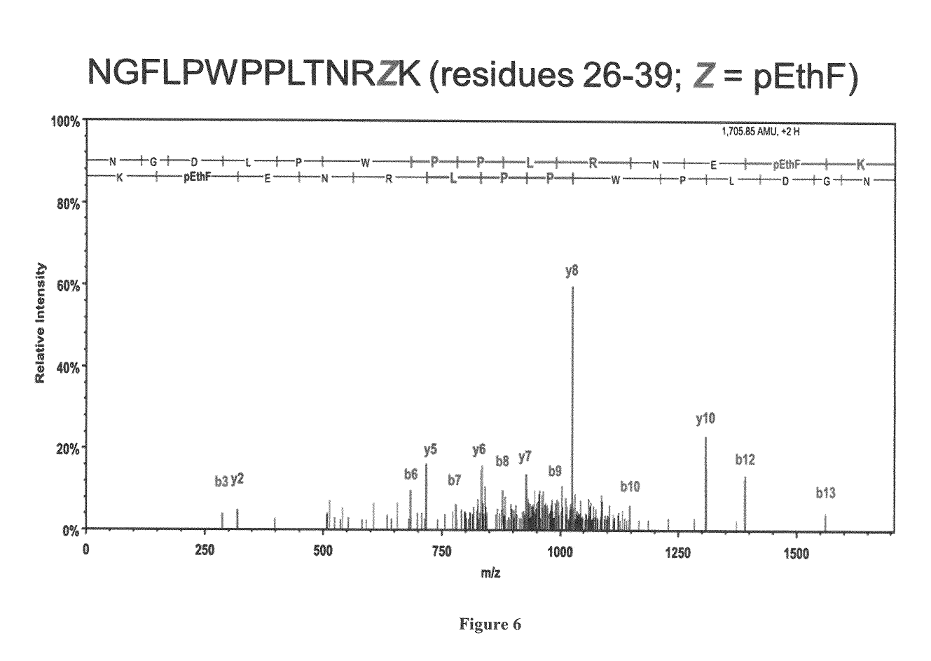

In order to investigate site-specific fatty acid-conjugation to a protein via CuAAC, we introduced pEthF into murine dihydrofolate reductase (mDHFR) in a site-specific manner. Since the expression and purification of DHFRs in E. coli are well established (38, 39), we routinely used mDHFR to study site-specific incorporation of a NAA. pEthF is a phenylalanine analog with an alkyne moiety at para-position of the phenyl ring (FIG. 1) and expected to act as a molecular handle for CuAAC with azide-functionalized molecules. Previously the yeast-originated pair of phenylalanine-tRNA suppressor/phenylalanyl-tRNA synthetase (ytRNA^{Phe}_{CIA}_{_{—}}_{UG}/yPheRS^{T415G}) was designed to incorporate 2-naphthylalanine, a Phe analog, into mDHFR in E. coli expression system (40). The relaxed substrate specificity of yPheRS^{T415G} also allows recognition of a panel of Phe and Trp analogs with a bulky functional group at para-position of the phenyl ring. Therefore, we hypothesize that the ytRNA^{Phe}_{CUA}_{UG}/yPheRS^{T415G} pair will also allow efficient site-specific incorporation of pEthF in response to an amber codon at the 38^{th} position of the mDHFR mutant (mDHFR-38Am). The mDHFR-38Am was expressed in the presence of 3 mM pEthF, purified under denaturing condition, and trypsin-digested for MALDI-TOF analysis as described previously with minor alterations (38, 39). The mDHFR mutant containing pEthF at the 38^{th} position is designated as mDHFR-pEthF. For wild-type mDHFR (mDHFR-WT), Peptide F38 (residues 26-39), one of tryptic digests, was detected with a monoisotopic mass of 1682.7 Da, in accord with its theoretical mass (FIG. 2A). Peptide Z38 of the mDHFR-pEthF (residues 26-39) was detected with a strong signal at a mass of 1706.8 Da, supporting the incorporation of pEthF in response to the amber codon. Furthermore, liquid chromatography—tandem mass spectrometry confirmed this assignment (FIG. 6).

To validate orthogonal reactivity of the alkyne end group of pEthF with an azide moiety via CuAAC, fluorogenic coumarin azide was reacted with the purified mDHFR-pEthF or mDHFR-WT in a CuSO₄/ascorbate system. Since reaction of the coumarin azide with an alkyne group produces a strongly fluorescent triazole-linked conjugate (41), the evolution of fluorescence is an indicator of pEthF reactivity for CuAAC. In SDS-PAGE analysis, the protein gel under UV exposure (λₑₓ=390 nm) clearly exhibited the fluorescence confirming the formation of a triazole linkage between coumarin azide and the alkynyl group of the mDHFR-pEthF (FIG. 2B) as well as a strong protein band stained with Coomassie blue dye, whereas the mDHFR-WT did not exhibit any fluorescence despite a strong protein band stained with Coomassie blue dye. The combined results of the mass spectrometric analysis and the fluorogenic dye conjugation strongly support the idea that pEthF was site-specifically incorporated into a protein using the E. coli expression system containing the orthogonal pair of ytRNA^{Phe}_{CUA}_{_{—}}_{UG}/yPheRS^{T415G}, and the pEthF introduced into a protein is reactive for bio-orthogonal CuAAC.

Site-Specific Fatty Acid-Conjugation to the mDHFR-pEthF

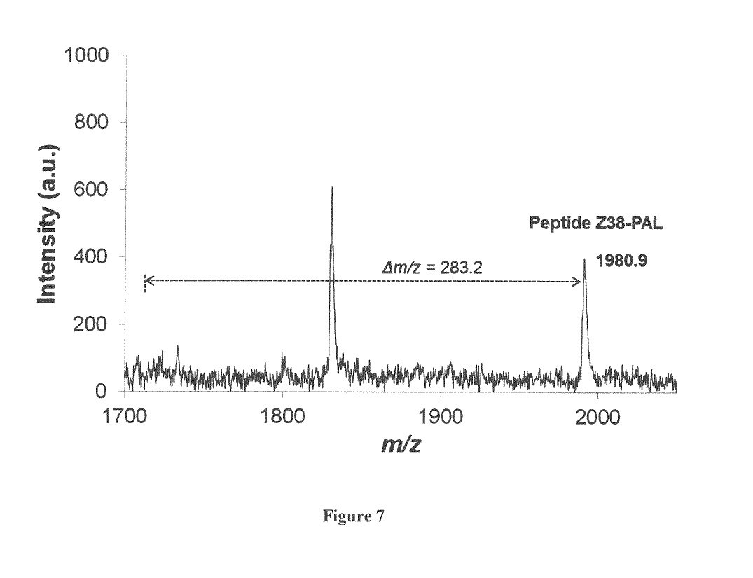

Next, we tested if a fatty acid with an intrinsic affinity for HSA can be grafted to the mDHFR-pEthF through CuAAC. Palmitic acid-azide (15-azidopentadecanoic acid), a palmitic acid analog containing an azide moiety at the end of the carbon chain (FIG. 1), was used for this purpose. The mDHFR-pEthF was reacted with palmitic acid-azide, and then subjected to tryptic digestion. The MALDI-TOF mass spectrum of the tryptic digests shows that a new signal with a monoisotopic mass of 1989.9 appears whereas Peptide Z38 signal is substantially reduced (FIG. 7). Considering that palmitic acid-azide conjugation will add 283.4 daltons to the mass of Peptide Z38 (the actual mass shift of 283.2 in the spectrum), the new peak is considered as palmitic acid-conjugated Peptide Z38 (Peptide Z38-PAL). This result clearly indicates that palmitic acid-azide has been conjugated to the mDHFR-pEthF in a site-specific manner.

Site-Specific Fatty Acid-Conjugation to Superfolder Green Fluorescent Protein without Compromising its Folded Structure and Intrinsic Fluorescence

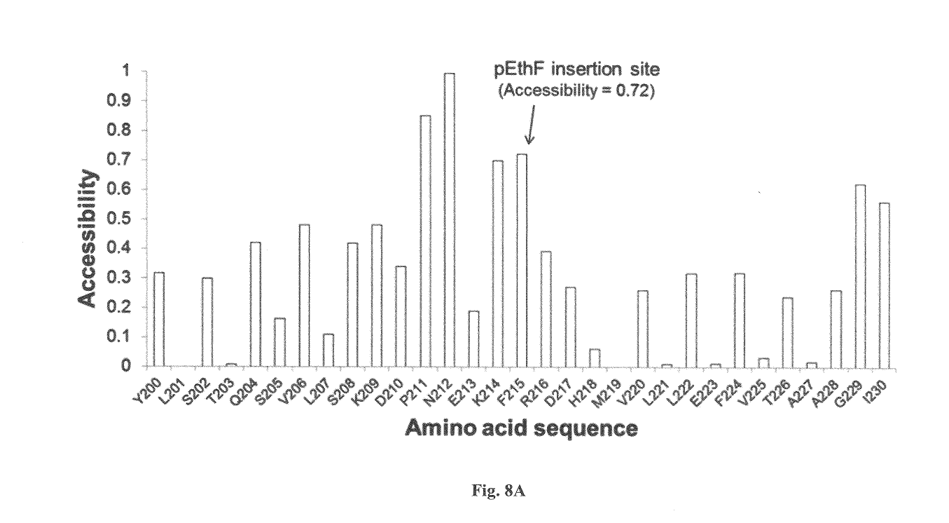

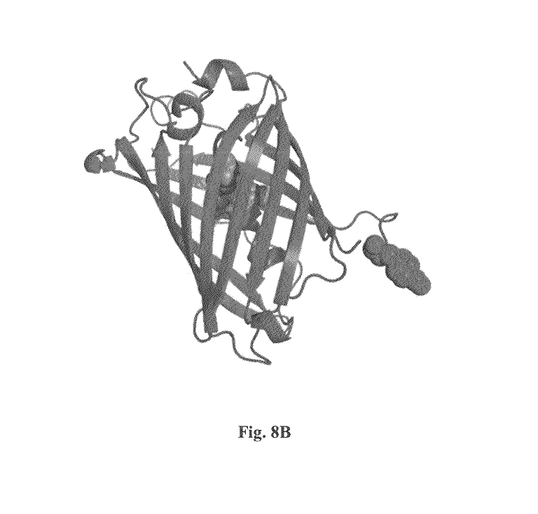

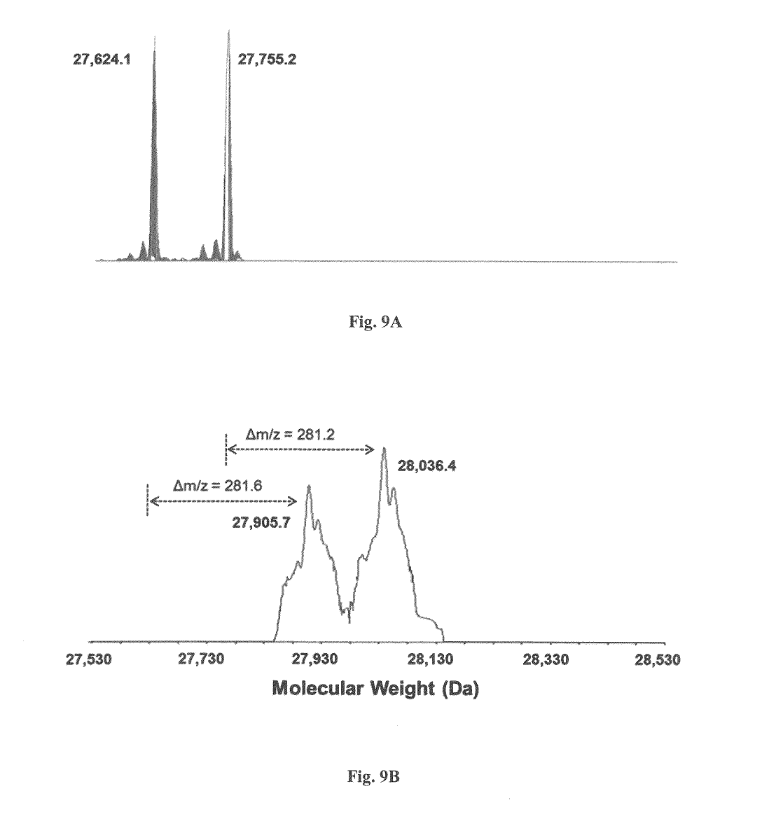

To investigate site-specific fatty acid-conjugation to a native protein without compromising intrinsic properties, we examined the site-specific fatty acid-conjugation to superfolder green fluorescent protein (sfGFP) (35). Intrinsic fluorescence of sfGFP correlated to its folding facilitates the estimation of the extent of structure perturbation during CuAAC and the determination of sfGFP quantity in the following characterization steps. In order to allow efficient fatty acid-conjugation with least protein structure perturbation, we chose a position between the 214^{th} and the 215^{th} amino acid as pEthF incorporation site by using a server-based solvent accessibility calculation program (ASA-View) (42) and examining the crystal structure of sfGFP. This position is located in a loop region with high solvent accessibility (0.72 score in the ASA-View) and distal from the chromophore (FIG. 8). Furthermore, it was reported that another NAA at this position can be used for CuAAC (26). pEthF was introduced into the amber codon site using the E. coli expression host harboring ytRNA^{Phe}_{CUA}_{_{—}}_{UG}/yPheRS^{T415G} orthogonal pair. The sfGFP variant containing pEthF at position 215 (sfGFP-pEthF) was purified via metal-ion affinity chromatography using a six-histidine tag. Based on the expression medium volume, about 80 mg/L of purified sfGFP-pEthF was obtained. Site-specific incorporation of pEthF into wild-type sfGFP (sfGFP-WT) was confirmed by mass spectrometry coupled with electron spray ionization (ESI-MS) (FIG. 9). The measured mass of the full length sfGFP variant (sfGFP-pEthF) is 27,755.2 Da, which is consistent with the calculated mass of 27,755.8 Da. The sfGFP-pEthF was then subjected to CuAAC-mediated palmitic acid-conjugation in a native condition using a CuSO₄/dithiothreitol (DTT)/Tris[(1-benzyl-1H-1,2,3-triazol-4-yl)methyl]amine (TBTA) system where TBTA acts as an accelerating ligand as well as a radical scavenger (23). The palmitic acid-conjugate (sfGFP-Pal) was analyzed by ESI-MS and found to have a molecular weight greater than that of the sfGFP-pEthF by 281.2 Da, strongly indicating site-specific conjugation at a stoichiometry of one palmitic acid per protein. As reported previously (43, 44), the N-terminal methionine was cleaved in a portion of the sfGFP-pEthF (ΔM-sfGFP-pEthF) generating a peak with 27,624.1 m/z (FIG. 9). However, an additional peak corresponding to the palmitic acid-conjugated AM-sfGFP-pEthF (27,905.7 m/z) (SI Fig. S4) was also detected, indicating that both the intact and the methionine-cleaved sfGFPs are successfully conjugated to the palmitic acid.

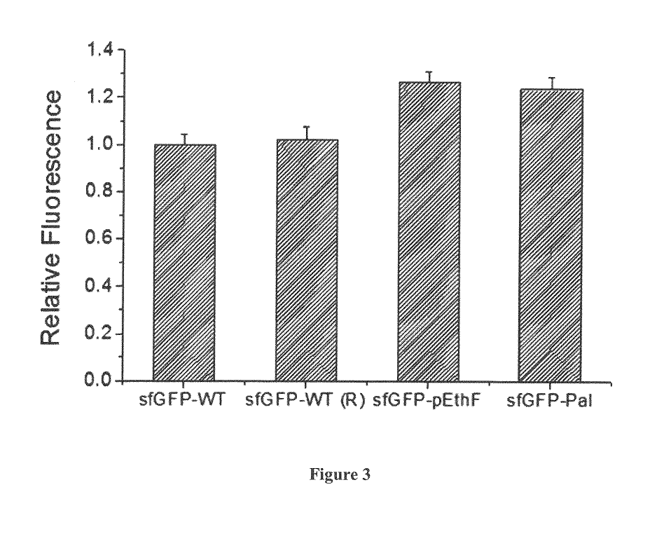

Next, we investigated whether the fatty acid-conjugation perturbs the sfGFP folded structure. As an indicator of the portion of correctly folded sfGFPs, the fluorescence intensity of sfGFPs was monitored (FIG. 3). The fluorescence intensity of the sfGFP-pEthF is 25% higher than that of the sfGFP-WT. Even after the sfGFP-pEthF was conjugated to a fatty acid, the fluorescence intensity of the sfGFP-Pal remains unchanged. Similarly, the incubation of the sfGFP-WT under the same condition used for the fatty acid-conjugation of the sfGFP-pEthF did not significantly alter the fluorescence intensity (FIG. 3). These results clearly demonstrate that neither the site-specific incorporation of pEthF at position 215 of sfGFP nor the fatty acid-conjugation via CuAAC compromises the intrinsic fluorescence and thereby folded structure of sfGFP.

HSA-Binding of the sfGFP-Pal In Vitro

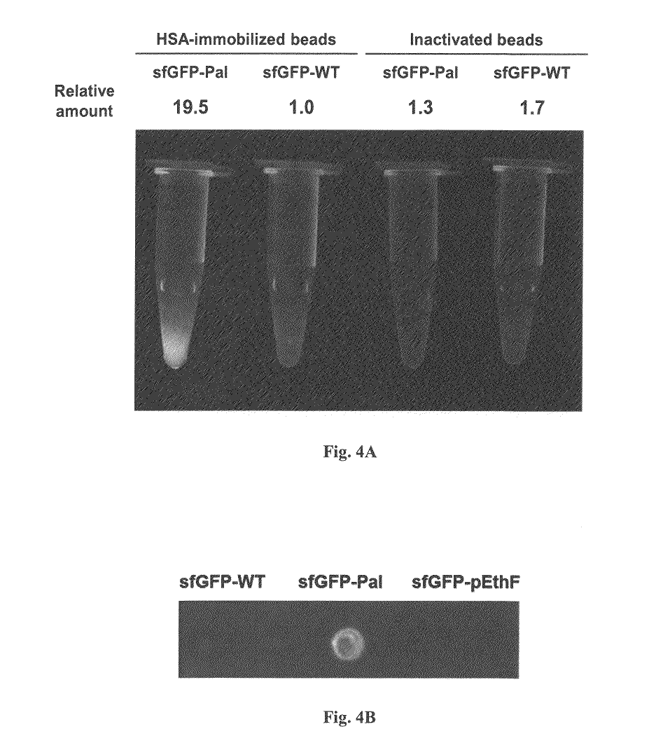

To investigate whether the fatty acid-conjugation to a protein generates albumin-binding affinity, the sfGFP-WT, and the sfGFP-Pal were mixed with HSA-coupled agarose beads or, as a control, inactivated beads in which amine-reactive N-hydroxysuccinimide groups had been blocked by glycine. After washing the beads multiple times with PBS on a gravity-flow column, the fluorescence intensity of the beads were qualitatively or quantitatively analyzed by using a fluorescence imager and a fluorescence microplate reader, respectively (FIG. 4). The HSA-coupled beads mixed with the sfGFP-Pal exhibit significant fluorescence while the HSA-coupled beads mixed with the sfGFP-WT display negligible fluorescence (FIG. 4A). Furthermore, the inactivated beads mixed with the sfGFP-Pal exhibit negligible fluorescence. These results strongly support the idea that the sfGFP-Pal binds the HSA-coupled beads via HSA-specific interactions. In order to quantitatively compare the binding affinities of the sfGFP-WT and the sfGFP-Pal to HSA, the fluorescence intensities of the HSA-coupled beads mixed with the sfGFP-WT and the sfGFP-Pal were measured. The HSA-coupled beads mixed with the sfGFP-Pal exhibit about 20-fold greater fluorescence than those with the sfGFP-WT. The inactivated beads mixed with the sfGFP-WT and the sfGFP-Pal exhibit 1.7- and 1.3-fold greater fluorescence than that of the HSA-coupled beads mixed with the sfGFP-WT, likely because the sfGFP-WT and the sfGFP-Pal reacted with a small amount of residual amine-reactive N-hydroxysuccinimide groups in the inactivated beads (FIG. 4A). Without any significant structural perturbation, the fluorescence intensity is directly correlated to the amount of sfGFP. Therefore, these results suggest that the fatty acid-conjugation leads to substantial increase in albumin-binding affinity. To eliminate the possibility that the fluorescence increase is caused by aggregation of the sfGFP-Pal leading to its retention in the column during the HSA-coupled bead-binding assay, the binding was also examined using the nitrocellulose membrane blot which was subjected to extensive washing. In contrast to the sfGFP-WT and the sfGFP-pEthF that were washed away after being spotted on HSA-coated membrane, the sfGFP-Pal was tightly bound to HSA as confirmed using fluorescence image analysis (FIG. 4B). These results unambiguously indicate that the site-specific palmitic acid-conjugation remarkably enhances HSA-binding affinity of the sfGFP compared to the unmodified sfGFP.

Pharmacokinetic Study of the sfGFP-Pal

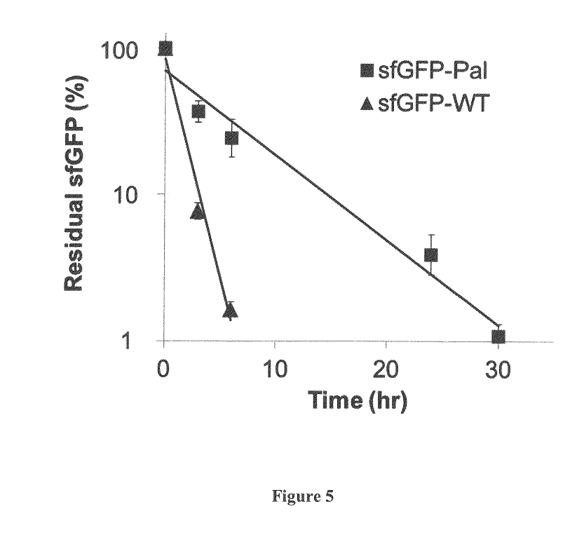

In order to evaluate clinical benefits from albumin-binding capacity generated from the site-specific fatty acid-conjugation, a single dose of either the sfGFP-WT or the sfGFP-Pal was intravenously administered to mice (n=4). The sfGFP concentrations in the serum samples taken at different time points were measured by using GFP-specific ELISA kit. Assuming one-compartment distribution and first-order elimination of the sfGFP in the serum (45, 46), its logarithmic residual serum concentrations versus time were plotted, and the data were fitted into a straight line to calculate the serum half-life (FIG. 5). The serum half-life of the sfGFP-Pal calculated (5.2 hr) is approximately 5-fold longer than that of the sfGFP-WT (1.0 hr).

### Discussion

The fatty acid-conjugation is an attractive methodology for developing long-acting protein therapeutics. Natural occurrence of a fatty acid in the blood greatly reduces the risk of immunogenicity and toxicity when it is used as an albumin-binding tag. In addition, its small size relative to other albumin-binding motifs (47, 48), including albumin-binding domain, is less likely to impair protein folded structure and function upon conjugation. The recently FDA-approved long-acting peptide analogs in which a fatty acid has been chemically linked to a lysine residue represent the potential of a fatty acid as a safe and reliable half-life extender in clinical settings. To render it broadly applicable to large-sized proteins as well as small peptides, new protein conjugation chemistry is required to prevent the production of positional isomers and detrimental loss of inherent activity arising from random coupling to multiple lysine residues.

Since its advent in 2002, CuAAC has found numerous applications in diverse fields, providing highly selective reactivity. To implement CuAAC for the site-specific attachment of a fatty acid to a protein, pEthF was introduced by using the engineered orthogonal pair of ytRNA^{Phe}_{CUA}_{_{—}}_{UG}/yPheRS^{T415G} with the yield of approximately 80 mg/L based on the volume of protein expression medium. Research efforts witnessed over the past couple of years have demonstrated near-optimal expression of a NAA-incorporated protein (up to 800 mg/L) comparable to that of its wild-type, thereby showing great promise for its expanded application to protein therapeutics (49-51). Successful bioconjugation via CuAAC is critically dependent on stabilizing catalytically active Cu(I) oxidation state while simultaneously preventing generation of reactive byproducts leading to undesirable protein aggregation. We discovered that the CuSO₄/DTT/TBTA system is suitable for the fatty acid-conjugation to a protein resulting in a high yield with minimal side products. The use of TBTA ligand was essential for a high yield and an optimal reaction rate, but its low solubility in water required the addition of a polar solvent, DMSO, in the CuAAC reaction. Newly-developed water-soluble ligands such as THPTA and BTTAA might be alternatives for bioconjugation of proteins intolerant to DMSO (52).

Disclosed herein is the utility of a fatty acid as an albumin-binding tag attached to a large protein with absolute site selectivity. Site-specificity is a critical key advantage of this new technique over other albumin-binding strategies relying on the genetic fusion of affinity motifs or random chemical attachment of synthetic binding molecules. Another key to exploiting this technology is imparting albumin-binding capability to a protein with minimal perturbation of its native activity and stability. As demonstrated herein, the site-specific fatty acid-conjugation via CuAAC does not cause any significant loss of the sfGFP fluorescence, strongly indicating that the native sfGFP structure was not perturbed. Based on examination of a crystal structure of a target protein and the solvent accessibility prediction, optimal sites for NAA incorporation and subsequent fatty acid-conjugation can be chosen, which has not been possible previously. Furthermore, the utility of this technology to modulate pharmacokinetics can be easily expanded by varying carbon chain lengths or by adding distinct chemical linkers between a fatty acid and a target protein. Tailoring the half-life of a therapeutic protein offers the advantage of being able to optimize the requirements of its intended clinical application (53, 54).

The animal study has clearly revealed the significance of albumin-binding effect on in vivo half-life extension. Five-fold longer retention of the sfGFP-Pal in blood compared to the sfGFP-WT is most likely attributed to FcRn-mediated recycling of the sfGFP-HSA complex, which is supported by in vitro HSA-binding assay. Previously, a GFP variant C-terminally attached to a PEG-like polymer exhibited only 2 hrs of serum half-life when injected intravenously into mice (55). Similarly, a single-chain diabody (scDb) C-terminally fused to an albumin-binding domain showed 2.6 hrs of serum half-life, despite the 13-fold half-life extension compared to that of unmodified one (56). Therefore, the half-lives of the GFP and the scDb conjugates were found to be smaller than that of the sfGFP-Pal (5.2 hr). Although differences in dose, concentration measurement, and data analysis complicate a direct comparison between half-life extension technologies, it is evident that the technique and approach described in this paper constitutes a significant impact on the optimization of therapeutic efficacy of a protein by virtue of unique features including immuno-safety and orthogonal chemistry unrestricted in site of modification, and thereby has broad applications to short-lived proteins.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

### BIBLIOGRAPHY

1. Aggarwal S R (2012) What's fueling the biotech engine-2011 to 2012. Nat Biotechnol 30(12):1191-1197.

2. Czajkowsky D M, Hu J, Shao Z, Pleass R J (2012) Fc-fusion proteins: new developments and future perspectives. EMBO Mol Med 4(10):1015-1028.

3. Kontos S, Hubbell J A (2012) Drug development: longer-lived proteins. Chem Soc Rev 41(7):2686-2695.

4. Kratz F, Elsadek B (2012) Clinical impact of serum proteins on drug delivery. J Control Release 161(2):429-445.

5. Chaudhury C, Brooks C L, Carter D C, Robinson J M, Anderson C L (2006) Albumin binding to FcRn: distinct from the FcRn-IgG interaction. Biochemistry 45(15):4983-4990.

6. Chaudhury C, et al. (2003) The major histocompatibility complex-related Fc receptor for IgG (FcRn) binds albumin and prolongs its lifespan. J Exp Med 197(3):315-322.

7. Sherman M R, Saifer M G, Perez-Ruiz F (2008) PEG-uricase in the management of treatment-resistant gout and hyperuricemia. Adv Drug Deliv Rev 60(1):59-68.

8. Libon C, et al. (1999) The serum albumin-binding region of streptococcal protein G (BB) potentiates the immunogenicity of the G130-230 RSV-A protein. Vaccine 17(5):406-414.

9. Stork R, Muller D, Kontermann R E (2007) A novel tri-functional antibody fusion protein with improved pharmacokinetic properties generated by fusing a bispecific single-chain diabody with an albumin-binding domain from streptococcal protein G. Protein Eng Des Sel 20(11):569-576.

10. Baggio L L, Huang Q, Brown T J, Drucker D J (2004) A recombinant human glucagon-like peptide (GLP)-1-albumin protein (albugon) mimics peptidergic activation of GLP-1 receptor-dependent pathways coupled with satiety, gastrointestinal motility, and glucose homeostasis. Diabetes 53(9):2492-2500.

11. Schulte S (2009) Half-life extension through albumin fusion technologies. Thromb Res 124 Suppl 2:S6-8.

12. Zhao H L, et al. (2007) Circumventing the heterogeneity and instability of human serum albumin-interferon-alpha2b fusion protein by altering its orientation. J Biotechnol 131(3):245-252.

13. Jonassen I, et al. (2012) Design of the novel protraction mechanism of insulin degludec, an ultra-long-acting basal insulin. Pharm Res 29(8):2104-2114.

14. Trussel S, et al. (2009) New strategy for the extension of the serum half-life of antibody fragments. Bioconjug Chem 20(12):2286-2292.

15. Havelund S, et al. (2004) The mechanism of protraction of insulin detemir, a long-acting, acylated analog of human insulin. Pharm Res 21(8):1498-1504.

16. Knudsen L B, et al. (2000) Potent derivatives of glucagon-like peptide-1 with pharmacokinetic properties suitable for once daily administration. J Med Chem 43(9):1664-1669.

17. Bhattacharya A A, Grune T, Curry S (2000) Crystallographic analysis reveals common modes of binding of medium and long-chain fatty acids to human serum albumin. J Mol Biol 303(5):721-732.

18. Spector A A (1975) Fatty acid binding to plasma albumin. J Lipid Res 16(3):165-179.

19. Pollaro L, Heinis C (2010) Strategies to prolong the plasma residence time of peptide drugs. Medchemcomm 1(5):319-324.

20. Buse J B, et al. (2011) Liraglutide treatment is associated with a low frequency and magnitude of antibody formation with no apparent impact on glycemic response or increased frequency of adverse events: results from the Liraglutide Effect and Action in Diabetes (LEAD) trials. J Clin Endocrinol Metab 96(6):1695-1702.

21. Zinman B, et al. (2012) Insulin degludec versus insulin glargine in insulin-naive patients with type 2 diabetes: a 1-year, randomized, treat-to-target trial (BEGIN Once Long). Diabetes Care 35(12):2464-2471.

22. Shechter Y, et al. (2012) Newly designed modifier prolongs the action of short-lived peptides and proteins by allowing their binding to serum albumin. Bioconjug Chem 23(8):1577-1586.

23. Chan T R, Hilgraf R, Sharpless K B, Fokin V V (2004) Polytriazoles as copper(I)-stabilizing ligands in catalysis. Org Lett 6(17):2853-2855.

24. Rostovtsev V V, Green L G, Fokin V V, Sharpless K B (2002) A stepwise huisgen cycloaddition process: copper(I)-catalyzed regioselective “ligation” of azides and terminal alkynes. Angew Chem Int Ed Engl 41(14):2596-2599.

25. Lallana E, Riguera R, Fernandez-Megia E (2011) Reliable and efficient procedures for the conjugation of biomolecules through Huisgen azide-alkyne cycloadditions. Angew Chem Int Ed Engl 50(38):8794-8804.

26. Bundy B C, Swartz J R (2010) Site-specific incorporation of p-propargyloxyphenylalanine in a cell-free environment for direct protein-protein click conjugation. Bioconjug Chem 21(2):255-263.

27. Patel K G, Swartz J R (2011) Surface functionalization of virus-like particles by direct conjugation using azide-alkyne click chemistry. Bioconjug Chem 22(3):376-387.

28. Budisa N (2004) Prolegomena to future experimental efforts on genetic code engineering by expanding its amino acid repertoire. Angew Chem Int Ed Engl 43(47):6426-6463.

29. Hohsaka T, Ashizuka Y, Taira H, Murakami H, Sisido M (2001) Incorporation of nonnatural amino acids into proteins by using various four-base codons in an Escherichia coli in vitro translation system. Biochemistry 40(37):11060-11064.

30. Jester B C, Levengood J D, Roy H, Ibba M, Devine K M (2003) Nonorthologous replacement of lysyl-tRNA synthetase prevents addition of lysine analogues to the genetic code. Proc Natl Acad Sci USA 100(24):14351-14356.

31. Link A J, Tirrell D A (2005) Reassignment of sense codons in vivo. Methods 36(3):291-298.

32. Liu C C, Schultz P G (2010) Adding new chemistries to the genetic code. Annu Rev Biochem 79:413-444.

33. Wang L, Brock A, Herberich B, Schultz P G (2001) Expanding the genetic code of Escherichia coli. Science 292(5516):498-500.

34. Zheng S, Kwon I (2012) Manipulation of enzyme properties by noncanonical amino acid incorporation. Biotechnol J 7(1):47-60.

35. Pedelacq J D, Cabantous S, Tran T, Terwilliger T C, Waldo G S (2006) Engineering and characterization of a superfolder green fluorescent protein. Nat Biotechnol 24(1):79-88.

36. Feng G, et al. (2000) Imaging neuronal subsets in transgenic mice expressing multiple spectral variants of GFP. Neuron 28(1):41-51.

37. Okabe M, Ikawa M, Kominami K, Nakanishi T, Nishimune Y (1997) ‘Green mice’ as a source of ubiquitous green cells. FEBS Lett 407(3):313-319.

38. Kwon I, Tirrell D A (2007) Site-specific incorporation of tryptophan analogues into recombinant proteins in bacterial cells. J Am Chem Soc 129(34):10431-10437.

39. Kwon I, Wang P, Tirrell D A (2006) Design of a bacterial host for site-specific incorporation of p-bromophenylalanine into recombinant proteins. J Am Chem

Soc 128(36):11778-11783.

40. Kwon I, Kirshenbaum K, Tirrell D A (2003) Breaking the degeneracy of the genetic code. J Am Chem Soc 125(25):7512-7513.

41. Beatty K E, Xie F, Wang Q, Tirrell D A (2005) Selective dye-labeling of newly synthesized proteins in bacterial cells. J Am Chem Soc 127(41):14150-14151.

42. Ahmad S, Gromiha M, Fawareh H, Sarai A (2004) ASAView: database and tool for solvent accessibility representation in proteins. BMC Bioinformatics 5:51.

43. Hirel P H, Schmitter M J, Dessen P, Fayat G, Blanquet S (1989) Extent of N-terminal methionine excision from Escherichia coli proteins is governed by the side-chain length of the penultimate amino acid. Proc Natl Acad Sci USA 86(21):8247-8251.

44. Wang F, Niu W, Guo J, Schultz P G (2012) Unnatural amino acid mutagenesis of fluorescent proteins. Angew Chem Int Ed Engl 51(40):10132-10135.

45. DiPiro J T (2010) Concepts in Clinical Pharmacokinetics (American Society of Health-System Pharmacists, Bethesda), 5th Ed, pp 1-12.

46. Yang Z, et al. (2004) PEGylation confers greatly extended half-life and attenuated immunogenicity to recombinant methioninase in primates. Cancer Res 64(18):6673-6678.

47. Dennis M S, et al. (2002) Albumin binding as a general strategy for improving the pharmacokinetics of proteins. J Biol Chem 277(38):35035-35043.

48. Tijink B M, et al. (2008) Improved tumor targeting of anti-epidermal growth factor receptor Nanobodies through albumin binding: taking advantage of modular Nanobody technology. Mol Cancer Ther 7(8):2288-2297.

49. Cho H, et al. (2011) Optimized clinical performance of growth hormone with an expanded genetic code. Proc Natl Acad Sci USA 108(22):9060-9065. 50. Johnson D B, et al. (2011) RF1 knockout allows ribosomal incorporation of unnatural amino acids at multiple sites. Nat Chem Biol 7(11):779-786.

51. Young T S, Ahmad I, Yin J A, Schultz P G (2010) An enhanced system for unnatural amino acid mutagenesis in E. coli. J MolBio1395(2):361-374.

52. Besanceney-Webler C, et al. (2011) Increasing the efficacy of bioorthogonal click reactions for bioconjugation: a comparative study. Angew Chem Int Ed Engl 50(35):8051-8056.

53. Schellenberger V, et al. (2009) A recombinant polypeptide extends the in vivo half-life of peptides and proteins in a tunable manner. Nat Biotechnol 27(12):1186-1190.

54. Nguyen A, et al. (2006) The pharmacokinetics of an albumin-binding Fab (AB.Fab) can be modulated as a function of affinity for albumin. Protein Eng Des Sel 19(7):291-297.

55. Gao W, Liu W, Christensen T, Zalutsky M R, Chilkoti A (2010) In situ growth of a PEG-like polymer from the C terminus of an intein fusion protein improves pharmacokinetics and tumor accumulation. Proc Natl Acad Sci USA 107(38):16432-16437.

56. Hopp J, et al. (2010) The effects of affinity and valency of an albumin-binding domain (ABD) on the half-life of a single-chain diabody-ABD fusion protein. Protein Eng Des Sel 23(11):827-834.

57. Takasu A, et al. (2011) Artificial extracellular matrix proteins containing phenylalanine analogues biosynthesized in bacteria using T7 expression system and the PEGylation. Biomacromolecules 12(10):3444-3452.

58. Arnold K, Bordoli L, Kopp J, Schwede T (2006) The SWISS-MODEL workspace: a web-based environment for protein structure homology modelling. Bioinformatics 22(2):195-201.

59. The PyMOL Molecular Graphics System, Version 1.2r1 Schrodinger, LLC.

What is claimed is:

1. A method for increasing serum half-life of a protein, the method comprising modifying the protein by incorporating a nonstandard amino into said protein and conjugating a fatty acid to said incorporated nonstandard amino acid, wherein said fatty acid has serum protein binding activity, thereby increasing the serum half-life of said protein.

2. The method of claim 1, wherein said incorporation is at a specific site of said protein.

3. The method of claim 1, wherein said nonstandard amino acid is a synthetic amino acid.

4. The method of claim 1, wherein the fatty acid is selected from the group consisting of palmitic acid, pentadecylic acid, margaric acid, stearic acid, nonadecylic acid, arachidic acid, heneicosylic acid, behenic acid, tricosylic acid, lignoceric acid, pentacosylic acid, linoleic acid, arachidonic acid, stearidonic acid, palmitoleic acid, vaccenic acid, paullinic acid, and oleic acid.

5. The method of claim 4, wherein the fatty acid is palmitic acid.

6. The method of claim 1, wherein said fatty acid comprises a reactive azido group.

7. The method of claim 1, wherein said fatty acid is conjugated to said incorporated nonstandard amino acid using a cycloaddition technique.

8. The method of claim 7, wherein the cycloaddition is copper-catalyzed alkyne-azide cycloaddition.

9. The method of claim 1, wherein said nonstandard amino acid is incorporated site-specifically.

10. The method of claim 1, wherein said nonstandard amino acid is inserted as an additional amino acid.

11. The method of claim 10, wherein said nonstandard amino acid is incorporated as a substitute amino acid.

12. The method of claim 10, where said nonstandard amino is incorporated using the orthogonal pair of yeast phenylalanyl-tRNA/phenylalanyl-tRNA synthetase.

13. The method of claim 12, wherein said nonstandard amino acid is p-ethynylphenylalanine.

14. The method of claim 1, wherein the nonstandard amino acid comprises a reactive alkyne group.

15. The method of claim 1, wherein when said modified protein comprising a nonstandard amino and a fatty acid conjugated to said nonstandard amino acid is administered to a subject, said conjugated fatty acid binds to a serum protein comprising fatty acid binding activity.

16. The method of claim 15, wherein said modified protein has increased binding affinity for a serum protein.

17. The method of claim 16, wherein said serum protein is selected from the group consisting of serum albumin or antibody.

18. The method of claim 1, wherein said protein is dihydrofolate reductase or superfolder green fluorescent protein.

19. The method of claim 1, wherein said protein is a therapeutic protein.

20. The method of claim 19, wherein said therapeutic protein is selected from the group consisting of cytokines and growth factors.

21. The method of claim 19, wherein said therapeutic protein is selected from the group consisting of EGF, PDGF, GCSF, IL6, IL8, IL10, MCP1, MCP2, Tissue Factor, FGFb, KGF, VEGF, PDGF, MMP1, MMP9, TIMP1, TIMP2, TGFI3, interferons, TNF-α, HGF, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, tumor necrosis factor-alpha and -beta, mullerian-inhibiting substance, mouse gonadotropin-associated peptide, inhibin, activin, vascular endothelial growth factor, integrin, thrombopoietin (TPO), nerve growth factors, NGF-beta, platelet-growth factor, transforming growth factors (TGFs), TGF-alpha and TGF-beta, insulin-like growth factor-I and -II, erythropoietin (EPO), osteoinductive factors, interferons, interferon-alpha -beta, and -gamma, colony stimulating factors (CSFs), macrophage-CSF (M-CSF), granulocyte-macrophage-CSF (GM-CSF), granulocyte-CSF (G-CSF), interleukins (ILs), IL-1, IL-1alpha, IL-2, IL-3, IL-4, IL-5, IL-6, IL-7, IL-8, IL-9, IL-10, IL-11, IL-12, IL-13, IL-14, IL-15, IL-16, IL-17, IL-18, LIF, G-CSF, GM-CSF, M-CSF, EPO, kit-ligand, FLT-3, angiostatin, thrombospondin, endostatin, neurotrophin, complement proteins, and LT.

22. A method of treating a disease, disorder, or injury, said method comprising administering to a subject in need thereof a pharmaceutical composition comprising an effective amount of a modified protein of claim 1, wherein said modified protein treats said disease, disorder, or injury.

23. The method of claim 22, wherein said protein is selected from the group consisting of EGF, PDGF, GCSF, IL6, IL8, IL10, MCP1, MCP2, Tissue Factor, FGFb, KGF, VEGF, PDGF, MMP1, MMP9, TIMP1, TIMP2, TGFI3, interferons, TNF-α, HGF, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, tumor necrosis factor-alpha and -beta, mullerian-inhibiting substance, mouse gonadotropin-associated peptide, inhibin, activin, vascular endothelial growth factor, integrin, thrombopoietin (TPO), nerve growth factors, NGF-beta, platelet-growth factor, transforming growth factors (TGFs), TGF-alpha and TGF-beta, insulin-like growth factor-I and -II, erythropoietin (EPO), osteoinductive factors, interferons, interferon-alpha -beta, and -gamma, colony stimulating factors (CSFs), macrophage-CSF (M-CSF), granulocyte-macrophage-CSF (GM-CSF), granulocyte-CSF (G-CSF), interleukins (ILs), IL-1, IL-1alpha, IL-2, IL-3, IL-4, IL-5, IL-6, IL-7, IL-8, IL-9, IL-10, IL-11, IL-12, IL-13, IL-14, IL-15, IL-16, IL-17, IL-18, LIF, G-CSF, GM-CSF, M-CSF, EPO, kit-ligand, FLT-3, angiostatin, thrombospondin, endostatin, neurotrophin, complement proteins, and LT.


SEQUENCE LISTING (2 sequences)

SEQ ID NO: 1 - DNA, 28 bp, Aequorea victoria
cccaacgaaaagtagcgtgaccacatgg

SEQ ID NO: 2 - DNA, 28 bp, Aequorea victoria
ccatgtggtcacgctacttttcgttggg